(19) United States
(12) Patent Application Publication
(10) Pub. No.: US 2026/0335584 A1
(43) Pub. Date: Oct. 29, 2026

(54) Longitudinal Hypoxic-Distress Alert Apparatus, System and Methodology

(71) Applicant: The Government of the United States, as Represented by the Secretary of the Army, Frederick, MD (US)

(72) Inventor: Beth Ann Beidleman, Holliston, MA (US)

(73) Assignee: The Government of the United States, as Represented by the Secretary of the Army, Frederick, MD (US)

(21) Appl. No.: 19/110,883

(22) PCT Filed: Jan. 31, 2024
(86) PCT No.: PCT/US2024/013706

§ 371 Date: Mar. 12, 2025

Related U.S. Application Data
(60) Provisional application No. 63/443,141, filed on Feb. 3, 2023.

(51) Int. Cl. A61M 16/10; A61B 5/00; A61B 5/1455

(52) U.S. Cl. CPC A61M 16/1005; A61B 5/14551; A61B 5/7275; A61B 5/746; A61B 2560/0431

(57) ABSTRACT

An apparatus, system and method for detecting a risk of Acute Mountain Sickness (AMS) in an individual includes a pulse arterial oxygen saturation (SpO₂) sensor configured to extract SpO₂ measurements from the individual and a portable computing device configured to be worn or carried by the individual and coupled to the SpO₂ sensor. The portable computing device includes a processor and an output device. The processor determines a Rate of Accumulated Hypoxic Debt (RAHD), a Rate of Accumulated Hypoxic Debt per Period (RAHDP) and a probability D that the individual has or will develop AMS, and compares the probability D to a first threshold to determine if treatment of the individual is needed.

### RELATED APPLICATION

This application claims the benefit of provisional application Ser. No. 63/443,141 filed Feb. 3, 2023 and titled “Longitudinal Hypoxic-Distress Alert System,” the entire content of which is hereby incorporated by reference.

### STATEMENT OF GOVERNMENT INTEREST

The invention described herein may be manufactured, used and licensed by or for the United States Government.

### BACKGROUND

The disclosure relates in general to hypoxic monitoring and in particular to prediction of hypoxemia, including Acute Mountain Sickness (AMS).

Deployment of individuals to altitudes of greater than 2500 meters may suffer from AMS and have symptoms including headache, dizziness, fatigue, nausea (vomiting) and sleep disturbances. Just one member of a team or group may jeopardize the entire mission.

U.S. Patent Application Publication No. US2020/0146602 published on May 14, 2020 entitled “Systems and Methods for Monitoring Subjects in Potential Hypoxic Distress” (hereinafter the “'602 Publication”) and its file wrapper contents are expressly incorporated by reference herein. The '602 Publication discloses a metric called Accumulated Hypoxic Debt (hereinafter “AHD”) in units of per-cent hours. AHD is calculated from real-time pulse arterial oxygen saturation (SpO₂) measurements extracted from an individual over time at altitude. In the '602 Publication, AHD is used as an independent variable in a longitudinal generalized linear mixed model to calculate the probability D that an individual is at risk of AMS during the first 24 hours at altitude. If the individual has a probability D between a first threshold and a second threshold that is greater than the first threshold, then the individual is treated by, for example, stopping the individual from ascending to a higher elevation. If the individual has a probability D greater than the second threshold, then the individual is treated by, for example, moving the individual to a lower elevation.

AHD is a universal metric that eliminates the need for other predictors such as altitude (SpO2 varies with altitude), physical exercise (desaturation due to physical exercise is captured by a lower SpO2), the use of medication (acetazolamide, for example, stimulates ventilation thereby increasing SpO2), and sex (higher SpO2 in women).

As disclosed in the '602 Publication, individual monitoring of AHD during altitude exposure is a promising approach for predicting altitude illness 4 to 8 hours before its occurrence (83% accurate), but sensitivity is low (70%).

Because of the sometimes dire consequences of AMS, including the possibility of high-altitude pulmonary edema and/or high-altitude cerebral edema as well as the potential threat to the overall mission of groups of individuals at high altitude, a need exists for a more accurate and higher sensitivity method and apparatus to predict AMS in individuals at altitude longer than 24 hours.

### BRIEF DESCRIPTION OF THE DRAWINGS

FIGS. 1A and 1B illustrate the difference in sleep saturations between healthy individuals and individuals with high-altitude pulmonary edema and/or high-altitude cerebral edema, in accordance with various embodiments of the disclosure.

FIG. 2 illustrates data of individuals participating in AMS research, in accordance with various embodiments of the disclosure.

FIG. 3 illustrates the prevalence of AMS versus time at altitude and the AHD as a function of time, in accordance with various embodiments of the disclosure.

FIGS. 4A-4C illustrates the Rate of Accumulated Hypoxic Debt per Period (RAHDP) metric, in accordance with various embodiments of the disclosure.

FIG. 5 illustrates the data collected during research, in accordance with various embodiments of the disclosure.

FIG. 6 illustrates the results of using RAHDP to predict AMS, in accordance with various embodiments of the disclosure.

FIG. 7 is a schematic diagram of one embodiment of an individualized hypoxia monitor, in accordance with various embodiments of the disclosure.

FIG. 8 illustrates application of a system for real-time, individualized prediction of hypoxemia at altitude, in accordance with various embodiments of the disclosure.

### DETAILED DESCRIPTION

Embodiments of the present disclosure will now be described with reference to the drawing figures, in which like reference numerals refer to like parts throughout.

In general, the inventive devices, systems, and methods described herein advantageously allow for the individual AMS status of an individual to be determined in real-time, communicated to the individual and one or more treatment plans to be communicated to the individual when the probability or risk of having AMS exceeds a threshold.

In accordance with the embodiments disclosed herein, an individualized real-time hypoxia monitoring device and system is disclosed that provides real-time situational awareness to protect individual health, optimize performance, and reduce injury at altitude and in enclosed spaces. Individual monitoring of AHD during altitude exposure is provided in which altitude illness can be predicted 4-8 hours before its occurrence with a greater than 85% accuracy and a greater than an 80% sensitivity in a diverse sample population over the first three days of exposure at altitude, such as exposure at 3600 meters for three days, for example. Prediction of AHD 4-8 hours prior to occurrence of AMS, i.e. prior to measuring that an individual has AMS) includes prediction of decrements in individual physical and cognitive performance, which may include headache, dizziness, fatigue, nausea (vomiting) and sleep disturbances and in more severe cases, confusion, shortness of breath at rest, chest tightness, impaired awareness and disturbed consciousness. Just one member of a team or group may jeopardize the entire mission.

Research indicates that a number of factors may contribute to the prevalence and severity of AMS, including the following:
Rate of ascent (the faster the ascent, AMS may be more severe)
Altitude attained (the higher the individual, AMS may be more severe)
Length of time at altitude, with AMS generally peaking after 18-24 hours at altitude if no further ascent
Degree of physical exertion may increase AMS risk
Medications such as Acetazolamide and Dexamethasone can lessen symptoms
Acclimatization status, with moderate altitude residents at less risk of AMS than lower altitude residents

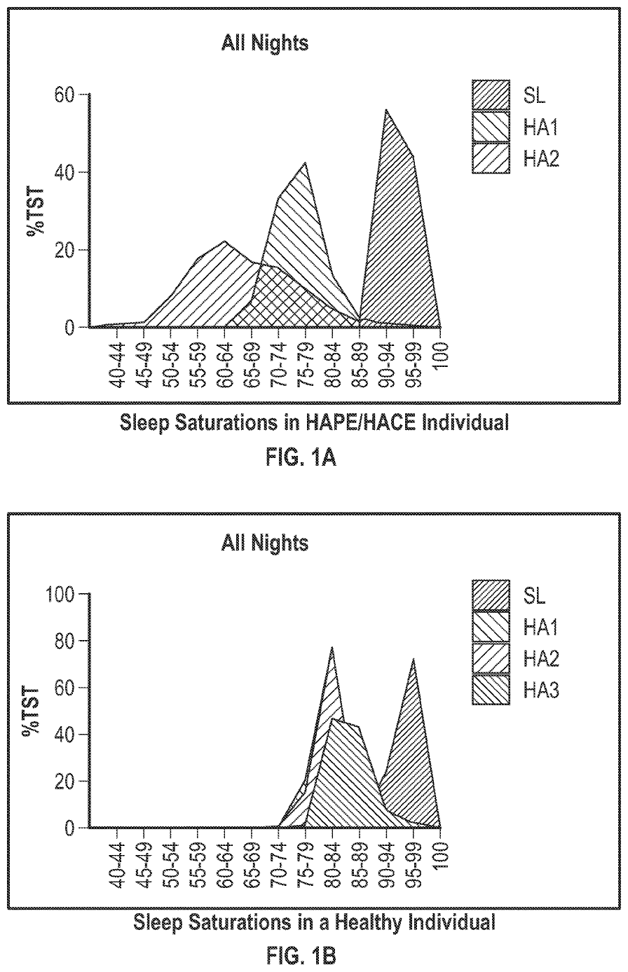

FIGa. 1A and 1B illustrates the difference in sleep saturations between healthy individuals and individuals with high-altitude pulmonary edema and/or high-altitude cerebral edema. Severe AMS can progress to life-threatening high-altitude pulmonary edema (HAPE) and/or high-altitude cerebral edema (HACE), both life-threatening conditions if not treated. In FIG. 1A, the sleep saturation of an individual in distress over days HA1 and HA2at altitude can be clearly seen, compared to the sleep saturations in a healthy individual over three days HA1, HA2, HA3 at altitude. The hypoxic monitoring apparatus and system with an AMS alert function would send a warning well before, as early as 8 hours for example, prior to the onset of HACE shown in FIG. 1A, providing more time for a safe evacuation and early treatment.

The disclosure includes apparatus and methods for real-time, individualized prediction of hypoxemia, including Acute Mountain Sickness (AMS). Because AMS has a time course that may peak around 20-24 hours and then decrease in a parabolic fashion, additional research was conducted to validate the methods disclosed in the '602 Publication when predicting AMS over a longer time period at altitude, for example, three days.

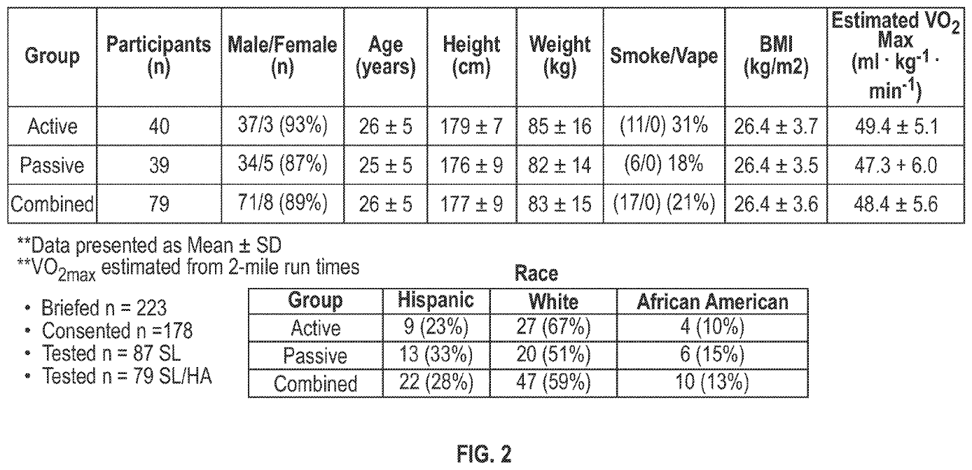

Additional research regarding the use of AHD as a predictor for AMS was conducted and included a larger sample size; a more diverse population, including women and low and moderate altitude residents; longer altitude exposure time; and both active and passive ascent. Subjects completing a study submitted to testing for a total of up to 68 hours including AMS and SpO₂ measurements, for example, during periods of activities at altitude, including an 800 meter ascent at altitude, and were compared to control individuals at altitude who did not undergo intense activity. The subjects were part of a diverse population, including men, women, low and moderate altitude residents, as shown in the consort diagram of FIG. 2 and were exposed for a longer altitude exposure time of up to 68 hours and to an altitude of 3600 meters. Additional energy expenditure of 1024+/−103 kcal was noted for active participants over passive participants.

In the '602 Publication, AHD was calculated using the “SpO₂ difference” where the SpO₂ difference was defined as 90% minus the average real-time SpO₂ calculated over a short time interval, for example 15 seconds. In accordance with the present disclosure, AHD was calculated using the SpO₂ difference where the SpO₂ difference is defined as 100% minus the average real-time SpO₂ calculated over a short time interval, for example 15 seconds. The SpO₂ difference is then multiplied by the length of the short time interval (for example, 15 seconds (15/3600 hours)) to obtain a single hypoxic debt amount in % hours. Then, the single hypoxic debt amounts are summed over the total time interval during which SpO₂ measurements have been taken to thereby obtain the AHD in % hours.

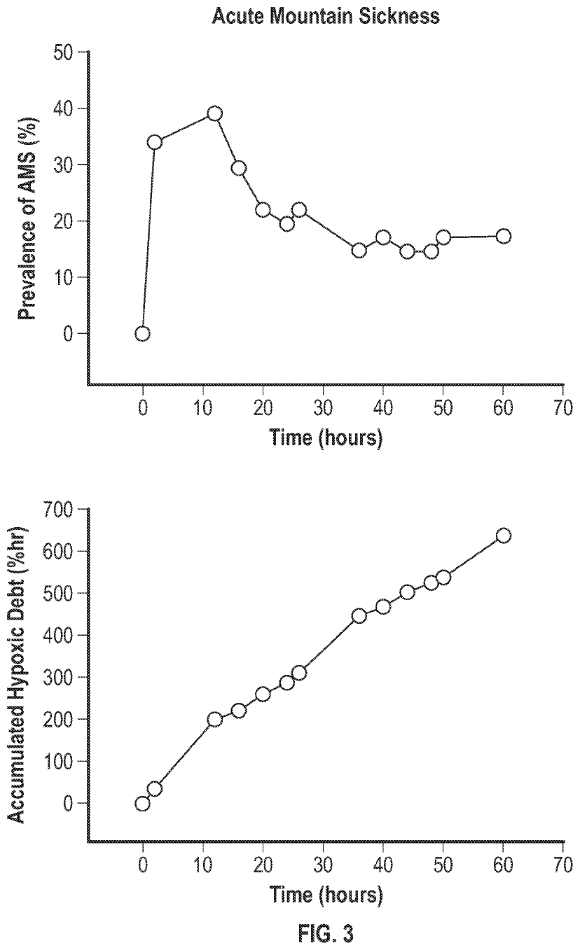

The graphs on FIG. 3 illustrate the prevalence of AMS versus time at altitude (upper graph) and the AHD (in per cent hour) (lower graph) as a function of time for 79 individuals at 3600 meters altitude for 63 hours. FIG. 3 shows that AHD alone may be a good predictor for one day at altitude, but its accuracy as a predictor decreases for an extended period at altitude, such as three days.

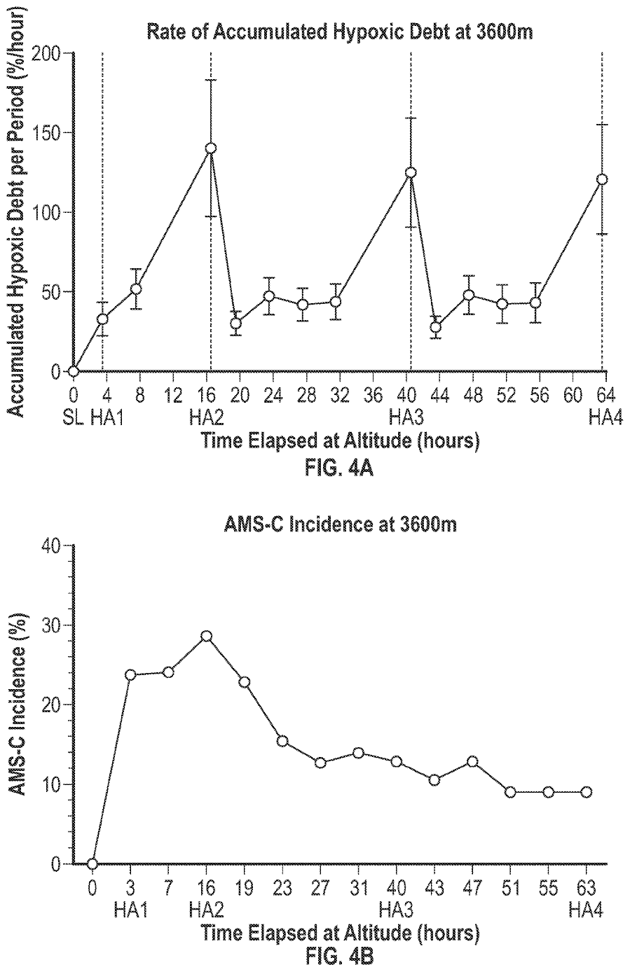

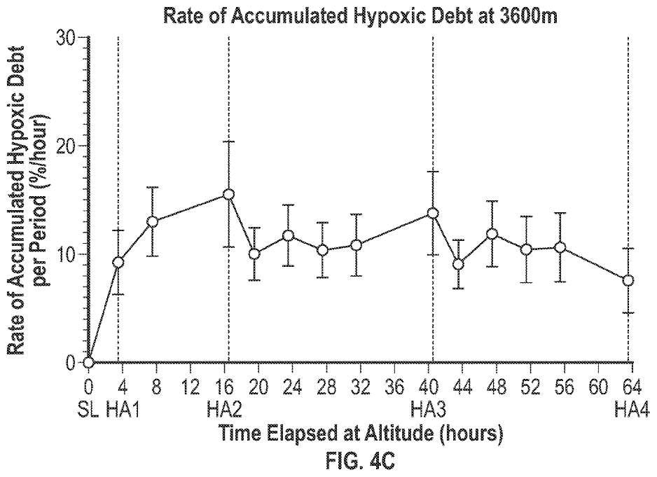

FIGS. 4A-4C illustrate a novel scientific finding about the rate of accumulated hypoxic debt per period (RAHDP), understood as the slope of the hypoxic debt accumulation in each period prior to an AMS measurement that confirms that the individual has AMS. In FIG. 4A, accumulated hypoxic debt, at 3600 meters in this example, does not fit the AMS-C curve, with P>0.05. In FIG. 4B, the AMS-C incidence at 3600 meters illustrates RAHDP*hours, with a P=0.0001. Finally, FIG. 4C illustrates the Rate of Accumulated Hypoxic Debt per Period (RAHDP) metric and shows the Rate of Accumulated Hypoxic Debt for the same 63 hours as in FIG. 3. In other words, the RAHDP fits the AMS curve of FIG. 3 and the relationship between RAHDP and time at altitude provides a new metric used to provide an individualized real-time hypoxia monitoring device and system that provides real-time situational awareness to protect individual health, optimize performance, and reduce injury at altitude.

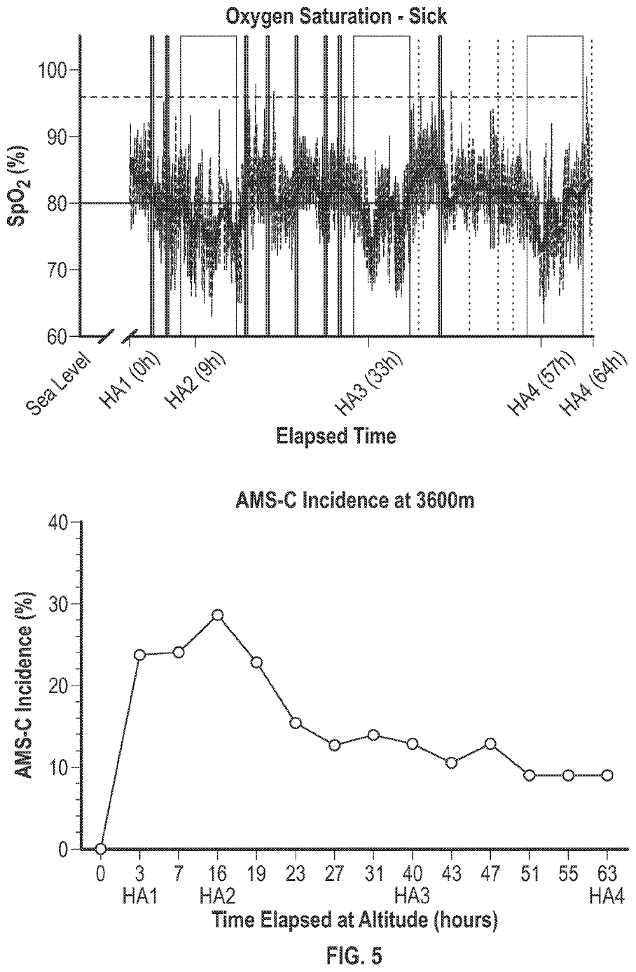

The location of the 13 data points on the x-axis in FIGS. 4A-4C correspond to the elapsed time period between each of the 13 self-assessments of AMS that were performed by each individual over 63 hours. The Rate of Accumulated Hypoxic Debt per Period (RAHDP) is the RAHD in each time period multiplied by the time length of the period. RAHD and RAHDP are novel metrics that may be used in a longitudinal generalized linear mixed model or equation to calculate the probability D that an individual has or is susceptible to AMS or is otherwise at risk of AMS when the individual is at altitude for a period of, for example, three days. Completed research in which 13 different measurements were taken every 4 seconds over 63 hours for 79 subjects yielded 57,150 measures/subject and a total of more than 4.5 million data points used to develop a prediction model. The model may predict AMS with a 91% or greater accuracy and a 78% or greater sensitivity. Some of the data points collected are illustrated in FIG. 5.

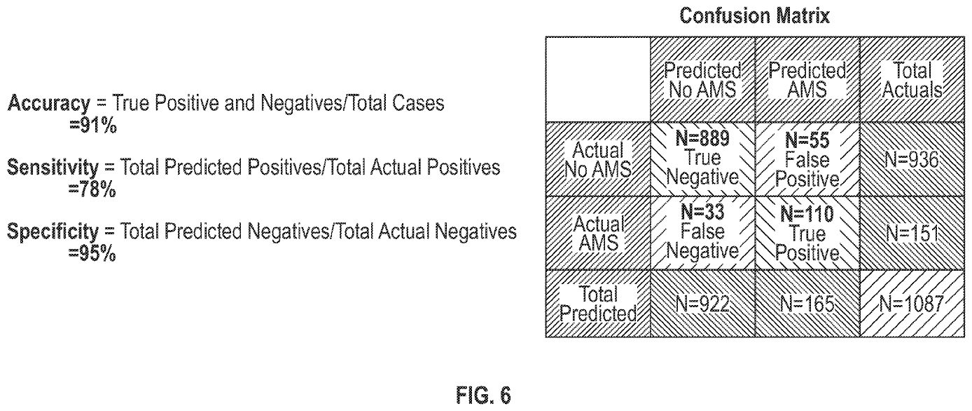

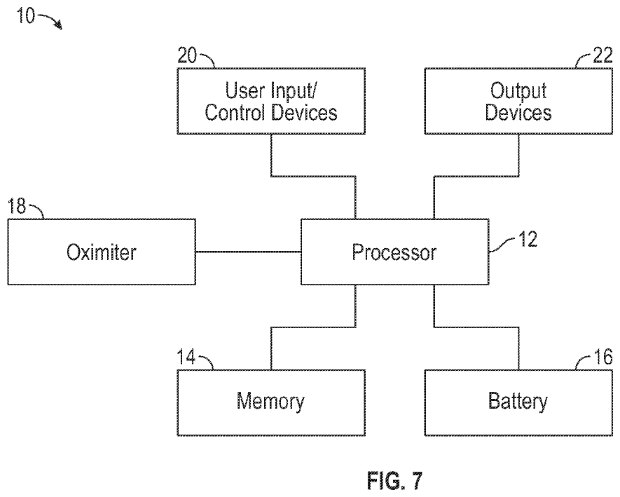

FIG. 6 illustrates using RAHDP in a system and apparatus to predict AMS. In this example, the accuracy defined as the (true positive and negatives) divided by the total cases is 91%, the sensitivity defined as the total predicted positives divided by the total actual positives is 78% and the specificity defined as the total predicted negatives divided by the total actual negatives is 95%. FIG. 7 is a schematic diagram of one embodiment of a real-time individualized hypoxia monitor or apparatus 10. Embodiments of the apparatus 10 may include a portable computing device, for example, a wrist-worn device and/or a smartphone, for example. Apparatus 10 either includes or is in communication with an oximeter (SpO₂ sensor) 18. Sensor 18 is coupled to a processor 12. Memory 14 and battery 16 (or other power supply) are coupled to the processor 12. User input and control devices 20 and output devices 22 are coupled to the processor 12. The user input and control devices 20 may include, for example, keyboards (virtual or real), touch screens, microphones, movable switches, ports and jacks, such as USB ports, memory card slots, such as SD card slots, etc. Output devices 22 may include, for example, visual displays, speakers, vibrating devices, antennas, ports and jacks, such as USB ports, memory card slots, such as SD card slots, etc.

Suitable SpO₂ sensors 18 suitable for receiving (extracting) from an individual are available from, for example, Equivital, Inc., Cambridge, UK; Athena GTX, Des Moines, IA: Masimo, Inc., Irvine, CA; and Nonin, Inc., Plymouth, MN. Headbands, such as an oximeter (Hox) headband, and patches may incorporate SpO₂ sensors (such as those by Lifelens, for example) embedded in various form, fit and function embodiments.

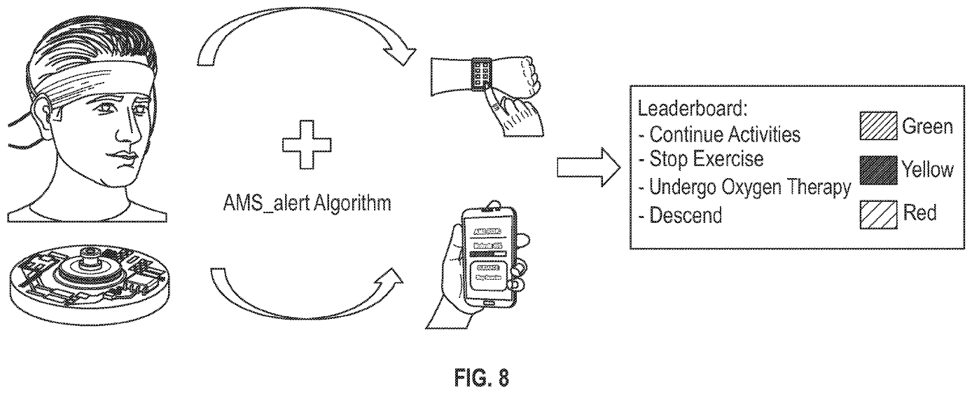

Portable and/or wearable devices may incorporate the required capabilities of processor 12, memory 14, battery 16 and input and output devices 20, 22. Wearable devices may include end user devices such as a smartphones, like a Motorola smartphone, a Samsung smartphone or an Apple iphone, and smart watches. Smart phones models would also be suitable and can be coupled to sensor 18 wirelessly via a Bluetooth® connection. Reference to FIG. 8 illustrates an example implementation of the teachings of the present disclosure. In this illustration, the user is shown wearing a wearable headband with the necessary sensors 18. Results determined by the AMS alert algorithm under controller of the processor are communicated to at least the individual user through a wearable device such as a smart watch or a smart phone.

It is not necessary that the individual user input any information into apparatus 10 other than the real-time SpO₂ measurements provided by sensor 18. Using the real-time SpO₂ measurements, the processor 12 determines the Accumulated Hypoxic Debt (AHD), the Rate of Accumulated Hypoxic Debt (RAHD), the Rate of Accumulated Hypoxic Debt per Period (RAHDP), and calculates the probability D of the individual experiencing AMS, developing AMS, at risk of developing AMS or is otherwise susceptible to AMS based upon these values. It is important to note that even if a human being could manually perform the calculations performed by the processor 12, the time required to manually calculate the real-time RAHDP at multiple time intervals and then manually calculate the probability D of experiencing AMS using would be prohibitive and would so interfere with the individual's other activities as to make doing so virtually worthless. Thus, a computing processor is a necessary and integral component of apparatus 10. Further, a software application or app resident on a wearable or portable device 10 may be operable to perform the processing performed by processor 12 described above.

Apparatus 10 may provide to the individual user their AMS status determined by their probability D via an output device 22, such as a visual display. In addition, the apparatus 10 may provide a visual indication of the category of the individual's AMS status determined by probability D, such as MILD, MODERATE, or SEVERE, for example. In addition to or as a substitute for a textual display, apparatus 10 may visually display a color code, such as green for MILD, yellow for MODERATE, or red for SEVERE. In some embodiments, apparatus 10 may provide instructions and appropriate courses of action to the individual, for example, on a visual display. For example, when the probability D is less than 30%, then a message such as “CONTINUE ACTIVITIES” that communicates the AMS status of the individual may be displayed; green for MILD may also be displayed. When probability D is more than 30% but less than 50%, then message(s) such as “STOP ASCENDING”, “STOP EXERCISE” and “UNDERGO OXYGEN THERAPY” or the like may be displayed; yellow for MODERATE may also be displayed. When probability D is more than 50%, then a message such as “DESCEND IMMEDIATELY” may be displayed; red for SEVERE may also be displayed. In one embodiment, when the probability D is greater than 30%, then a change in the individual's level of activity is indicated. Other instructions and other ranges of probabilities may be used. FIG. 8 shows example alerts that may displayed to at least an individual through a wearable device the individual is wearing, such as a smart phone or a smart watch.

For example, when the individual has a probability D between a first threshold and a second threshold that is greater than the first threshold (for example, between 30% and 50%), then the individual may be treated by stopping the individual from ascending to a higher elevation. In addition, when the individual has a probability D between the first threshold and the second threshold, the individual may be further treated by administering Acetazolamide, Dexamethasone and/or administering oxygen therapy. When the individual has a probability D greater than the second threshold, the individual may be treated by moving the individual to a lower elevation.

Embodiments of the disclosure have been described to explain the nature of the innovation. Those skilled in the art may make changes in the details, materials, steps and arrangement of the described embodiments within the principle and scope of the disclosure, as expressed in the appended claims. While implementations of the disclosure are susceptible to embodiment in many different forms, there is shown in the drawings and will herein be described in detail specific embodiments, with the understanding that the present disclosure is to be considered as an example of the principles of the disclosure and not intended to limit the disclosure to the specific embodiments shown and described. In the description above, like reference numerals may be used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” “includes,” “including,” “has,” “having,” or any other variations thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C.” An exception to this definition will occur only when a combination of elements, functions, steps or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

In the following description, it is understood that terms such as “first,” “second,” “top,” “bottom,” “up,” “down,” “above,” “below,” and the like, are words of convenience and are not to be construed as limiting terms. Also, the terms apparatus, device, system, etc. may be used interchangeably in this text.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A method of detecting and treating Acute Mountain Sickness (AMS) in an individual, comprising:
determining a Rate of Accumulated Hypoxic Debt (RAHD) and a Rate of Accumulated Hypoxic Debt per Period (RAHDP) of the individual based on SpO₂ measurements of the individual for a plurality of periods of measurement;
determining a probability D that the individual has or is susceptible to AMS, based on the RAHD and the RAHDP, and an AMS status of the individual based on the determined probability D;
communicating the AMS status of the individual and when the probability D of the individual is greater than a first threshold communicating a treatment for the individual.

2. The method of claim 1, where when the probability D of the individual is between the first threshold and a second threshold that is greater than the first threshold, further comprising communicating to the individual to stop ascending to a higher elevation; and
when the probability D of the individual is greater than the second threshold, then further comprising communicating to the individual to move to a lower elevation.

3. The method of claim 2, where when the probability D of the individual is between the first threshold and the second threshold, further comprising treating the individual by administering acetazolamide or dexamethasone.

4. The method of claim 2, where when the probability D of the individual is between the first threshold and the second threshold, further comprising treating the individual by administering oxygen therapy.

5. The method of claim 2, when the probability D of the individual is between the first threshold and the second threshold further comprising treating the individual by stopping the individual from ascending to the higher elevation and when the probability D of the individual is greater than the second threshold further comprising treating the individual by moving the individual to a lower elevation.

6. The method of claim 1, further comprising:
receiving a plurality of real-time SpO₂ measurements of the individual for a plurality of periods; and
determining the RAHD and the RAHDP of the individual from the SpO₂ measurements of the individual.

7. The method of claim 6, where the RAHDP is the slope of the hypoxic debt accumulation for the plurality of periods measured.

8. The method of claim 7, where the determining the RAHD and the RAHDP of the individuals from the SpO₂ measurements of the individual, the determining the probability D that the individual has AMS and the AMS status of the individual based on the determined probability D, and the communicating the AMS status of the individual based on the probability D is performed by a portable computing device receiving the plurality of real-time SpO₂ measurements of the individual.

9. The method of claim 8, further comprising the portable computing device communicating the treatment for the individual when the probability D of the individual is greater than the first threshold.

10. The method of claim 8, further comprising a pulse arterial oxygen saturation (SpO₂) sensor measuring the SpO₂ measurements from the individual in real-time and communicating them to the portable computing device.

11. The method of claim 1, further comprising:
the determining the RAHD and the RAHDP and the determining the probability D occurring prior to measuring that the individual has AMS.

12. A system for detecting a risk of Acute Mountain Sickness (AMS) in an individual, comprising:
a pulse arterial oxygen saturation (SpO₂) sensor configured to extract SpO₂ measurements from the individual in real-time; and
a portable computing device configured to be worn or carried by the individual and communicatively coupled to the SpO₂ sensor, the portable computing device configured to receive the SpO₂ measurements from the SpO₂ sensor and including a processor and an output device,
where the portable computing device is operable to determine a Rate of Accumulated Hypoxic Debt (RAHD) and a Rate of Accumulated Hypoxic Debt per Period (RAHDP) based on the received SpO₂ measurements, determine a probability D that the individual has or is susceptible to AMS based on the RAHD and the RAHDP, compare the probability D to at least a first threshold to determine a AMS status of the individual, communicate the AMS status of the individual, and communicate a treatment for the individual when the probability D of the individual is greater than a first threshold.

13. The system of claim 12, where the processor communicates the treatment to the individual via the output device.

14. The system of claim 13, where, when the probability D of the individual is between the first threshold and a second threshold that is greater than the first threshold, then the portable computing device communicates to the individual that the treatment is stopping the individual from ascending to a higher elevation; and when the probability D of the individual is greater than the second threshold, then the treatment is moving the individual to a lower elevation.

15. The system of claim 14, where, when the probability D of the individual is between the first threshold and the second threshold, then the treatment is additionally administering acetazolamide or dexamethasone to the individual.

16. The system of claim 15, where, when the probability D of the individual is between the first threshold and the second threshold, then the treatment is additionally administering oxygen therapy to the individual.

17. The system of claim 12, where the portable computing device further includes a memory coupled to the processor and operable to store the SpO₂ measurements received from the SpO₂ sensor.

18. The system of claim 12, where the portable computing device receives from the SpO₂ sensor a plurality of real-time SpO₂ measurements of the individual for a plurality of periods and determines the RAHD and the RAHDP of the individual from the real-time SpO₂ measurements for the plurality of periods.

19. The system of claim 18, where the RAHDP is the slope of the hypoxic debt accumulation for the plurality of periods measured.

20. A method of detecting Acute Mountain Sickness (AMS) in a human, comprising:
obtaining SpO₂ measurements from the human;
detecting whether the human is at risk of AMS by transforming the SpO₂ measurements to a Rate of Accumulated Hypoxic Debt (RAHD) and a Rate of Accumulated Hypoxic Debt per Period (RAHDP) and determining a probability D that the human is at risk of AMS from the RAHD and the RAHDP, the probability D determined by processing the RAHD and the RAHDP in a longitudinal generalized linear mixed equation.

21. The method of claim 20, further comprising:
communicating an AMS status of the human determined from the probability D;
communicating a treatment for the individual when the probability D of the individual is greater than a first threshold.

22. The method of claim 20, further comprising:
the detecting whether the human is at risk of AMS and the determining the probability D occurring prior to measuring that the individual has AMS.

23. An apparatus for detecting and communicating a risk of Acute Mountain Sickness (AMS) in an individual, comprising:
a portable computing device configured to be worn or carried by the individual and including a processor and an output device,
where the portable computing device is operable to:
determine a Rate of Accumulated Hypoxic Debt (RAHD) and a Rate of Accumulated Hypoxic Debt per Period (RAHDP) based on a plurality of received SpO₂ measurements,
determine a probability D that the individual has or is susceptible to AMS based on the RAHD and the RAHDP,
compare the probability D to at least a first threshold to determine an AMS status of the individual,
communicate the AMS status of the individual, and
communicate a treatment for the individual when the probability D of the individual is greater than a first threshold.
